(19) United States
(12) Patent Application Publication
(10) Pub. No.: US 2003/0211549 A1
(43) Pub. Date: Nov. 13, 2003

(54) Functional proteins and therapeutic and diagnostic methods for use thereof

(76) Inventor: Andrew J. Murphy, Croton-On-Hudson, NY (US)

Correspondence Address: REGENERON PHARMACEUTICALS, INC, 777 OLD SAW MILL RIVER ROAD, TARRYTOWN, NY, 10591, US

(21) Appl. No.: 10/373,617

(22) Filed: Feb. 25, 2003

Related U.S. Application Data
(63) Continuation-in-part of application No. 09/684,197, filed on Oct. 6, 2000, now abandoned.

(51) Int. Cl. G01N 33/53; G01N 33/567; A01K 67/00; A61K 38/22; C07K 14/47; C12P 21/02; C12N 5/06

(52) U.S. Cl. 435/7.2; 435/69.1; 435/320.1; 435/325; 514/12; 530/350; 800/8; 536/23.5

(57) ABSTRACT

This invention relates to OGH BETA 14 proteins as well as methods of producing and using such proteins. The present invention provides a functional Oghbeta14 protein. The proteins of the invention are useful for decreasing body weight, decreasing body fat, decreasing serum cholesterol, decreasing triglycerides, decreasing blood glucose, and increasing adrenal medullary mass in a subject

### PRIORITY

The application claims priority to U.S. application Ser. No. 09/684,197, filed on Oct. 6, 2000. The disclosure of this application is hereby incorporated by reference into this application.

### BACKGROUND OF THE INVENTION

1. Field of the Invention

This invention relates to OGH BETA 14 proteins as well as methods of producing and using such proteins.

2. Description of Related Art

Hormones that signal via GPCRs include, but are not limited to, the tropic adenohypophyseal hormones follicle stimulating hormone (FSH), luteinizing hormone (LH), and thyroid stimulating hormone (TSH). In addition, chorionic gonadotropin (CG), which is secreted by a developing embryo following endometrial implantation, also signals through a GPCR. In fact, LH and CG signal through the same GPCR. All four hormones are glycoproteins and belong to the glycoprotein hormone-beta family. Each hormone is a heterodimer comprised of a common glycoprotein hormone alpha (a) subunit and a beta (b) subunit. All four family members share the same a subunit, but each has its own unique b subunit. The b subunit provides for binding specificity to each hormone's cognate GPCR. This family of polypeptide hormones represents an important class of molecules with functions ranging from reproduction, cellular metabolism, and growth and development to blood calcium and bone homeostasis.

### BRIEF SUMMARY OF THE INVENTION

The present invention provides a functional Oghbeta14 protein. The proteins of the invention are useful for decreasing body weight, decreasing body fat, decreasing serum cholesterol, decreasing triglycerides, decreasing blood glucose, and increasing adrenal medullary mass in a subject.

Accordingly, in a first aspect the invention provides a protein comprising an OGHbeta14 polypeptide. In one embodiment, the OGHbeta14 polypeptide comprises the amino acid sequence as set forth in SEQ ID NO:2 or 9. In another embodiment, the OGHbeta14 polypeptide encoded by the nucleic acid sequence as set forth in SEQ ID NO:1 or 8.

A second aspect of this invention provides a nucleic acid molecule having a sequence selected from the group consisting of: (a) the nucleotide sequence comprising the coding region of the OGHbeta14 as set forth in SEQ ID NO:1; or (b) a nucleotide sequence that hybridizes under stringent conditions to the complement of the nucleotide sequence of (a) and which encodes OGHbeta14, wherein said stringent conditions are 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE; or (a) a nucleotide sequence which, as a result of the degeneracy of the genetic code, differs from the nucleic acid of (a) or (b) and which encodes OGHbeta14.

In a third aspect, the invention features a method of decreasing weight, comprising administering a therapeutically effective amount of OGHbeta14 protein, or protein fragment to an animal.

In a fourth aspect, the invention features therapeutic method for treating obesity, comprising administering a therapeutically effective amount of OGHbeta14 protein, or protein fragment, to a subject in need thereof. In a preferred embodiment, the subject is a human patient.

In a fifth aspect, the invention features a therapeutic method for modulating feeding or metabolism, comprising administering a therapeutically effective amount of OGHbeta14 protein, or protein fragment, to a subject in need thereof.

In a sixth aspect, the invention features pharmaceutical compositions useful for treatment of obesity and related conditions, comprising OGHbeta14 and a pharmaceutical acceptable carrier.

In a seventh aspect, the invention features pharmaceutical compositions useful for decreasing weight in a subject in need thereof, comprising OGHbeta14 and a pharmaceutically acceptable carrier.

In an eighth aspect, the invention features pharmaceutical compositions useful for modulating feeding or metabolism comprising OGHbeta14 and a pharmaceutically acceptable carrier.

In a ninth aspect, the invention features a transgenic animal comprising a modification of an endogenous OGHbeta14 gene. As described more fully in co-pending U.S. Ser. No. 09/732,234 filed Dec. 7, 2000, the transgenic animal of the invention is generated by targeting the endogenous OGHbeta14 gene with a large targeting vector (LTVEC). In one embodiment of the transgenic animal of the invention, the animal is a knock-out wherein the OGHbeta14 gene is altered or deleted such that the function of the endogenous OGHbeta14 protein is reduced or ablated. In another embodiment, the transgenic animal is a knock-in animal modified to comprise an exogenous gene.

In a tenth aspect, the invention features a vector comprising a nucleic acid of the invention. The invention further features an expression vector comprising a nucleic acid of the invention, wherein the nucleic acid molecule is operably linked to an expression control sequence. Also provided is a host-vector system for the production of a protein of the invention which comprises the expression vector of the invention which has been introduced into a host cell suitable for expression of the protein. Suitable host cells include, for example, bacterial cells, e.g., E. coli, yeast cells, e.g., Pichia pastoris, an insect cell, e.g., Spodoptera frugiperda, or a mammalian cell, such as CHO or COS.

In an eleventh aspect, the invention features a method of producing a protein of the invention, comprising culturing a host cell transfected with a vector comprising a nucleic acid sequence of the invention, under conditions suitable for expression of the protein from the host cell, and recovering the protein so produced.

In a related twelfth aspect, the invention features a polypeptide produced by the culturing a host cell transfected with a vector comprising a nucleic acid sequence of the invention, under conditions suitable for expression of the protein from the host cell. A composition may comprising OGHbeta14 and a carrier. Such polypeptide composition may be used in a method of treatment of the human or animal body, or in a method of diagnosis.

In a thirteen aspect, the invention comprises a method for identifying agents capable of binding a OGHbeta14 protein, or protein fragment, comprising (a) contacting a test agent with a OGHbeta14 protein, or protein fragment; and (b) determining the ability of the test agent to bind OGHbeta14 protein or protein fragment. In this aspect the test agent may be a receptor for OGHbeta14. The receptor may be a GPCR and/or may be involved in metabolism or feeding. The method may utilize the OGHbeta14 protein comprising the amino acid sequence of SEQ ID NO:2 or 9, or a fragment thereof. The screening methods of the invention include in vitro and in vivo assays.

In one embodiment of an in vitro screening method of the invention, agents capable of binding the OGHbeta14 protein or protein fragment are identified in a cell-based assay system. More specifically, cells expressing a potential OGHbeta14 protein are contacted with OGHbeta14, and the ability of the candidate cell to bind OGHbeta14 or a fragment thereof is determined.

In another embodiment, agents capable of binding a OGHbeta14 protein or protein fragment are identified in a cell-free assay system. More specifically, a native or recombinant human OGHbeta14 protein or protein fragment is contacted with a candidate compound or a control compound, and the ability of the candidate compound to bind OGHbeta14 or a fragment thereof is determined.

In another embodiment, agents capable of binding OGHbeta14 or a fragment thereof are identified in vivo in an animal system. More specifically, a candidate agent or a control compound is administered to a suitable animal, and the effect on OGHbeta14-mediated weight regulation and/or fluctuation, obesity, feeding and/or metabolism is determined. Any suitable assay known to the art for determination of these activities, for example measurement of body mass and telekenetics, may be used.

In a fourteenth aspect, the invention features diagnostic and prognostic methods, as well as kits for detecting, quantitating, and/or monitoring obesity, feeding, metabolism and/or weight through the use of the proteins of the invention.

Other objects and advantages will become apparent from a review of the ensuing detailed description.

### BRIEF DESCRIPTION OF THE FIGURES

FIG. 1 (SEQ ID NOs: 1 and 2) shows the nucleic acid sequences (sense and antisense) (SEQ ID NO: 1) and deduced amino acid sequence of HUMAN OGHbeta14 (SEQ ID NO: 2).

FIG. 2 (SEQ ID NOs: 3,4,5,6, and 7) shows a sequence comparison of the novel b subunit polypeptide identified as HUMAN OGHbeta14 (SEQ ID NO: 6) with the b subunits of the other family members: rLH-beta (SEQ ID NO: 3), hCG-beta-e (SEQ ID NO: 4), rTSH-beta (SEQ ID NO: 5), and rFSH-beta (SEQ ID NO: 7). Asterisks (*) indicate amino acid identity between all family members.

FIG. 3 (SEQ ID NOs: 8 and 9) shows the nucleic acid sequences (sense and antisense) (SEQ ID NO: 8) and deduced amino acid sequence of HUMAN OGHbeta14 including additional 5′ sequence (SEQ ID NO: 9).

FIG. 4 (SEQ ID NOs: 10 and 11) shows nucleic acid (sense and antisense) (SEQ ID NO: 10) and amino acid sequences (SEQ ID NO: 11) of the common glycoprotein hormone alpha (α) subunit.

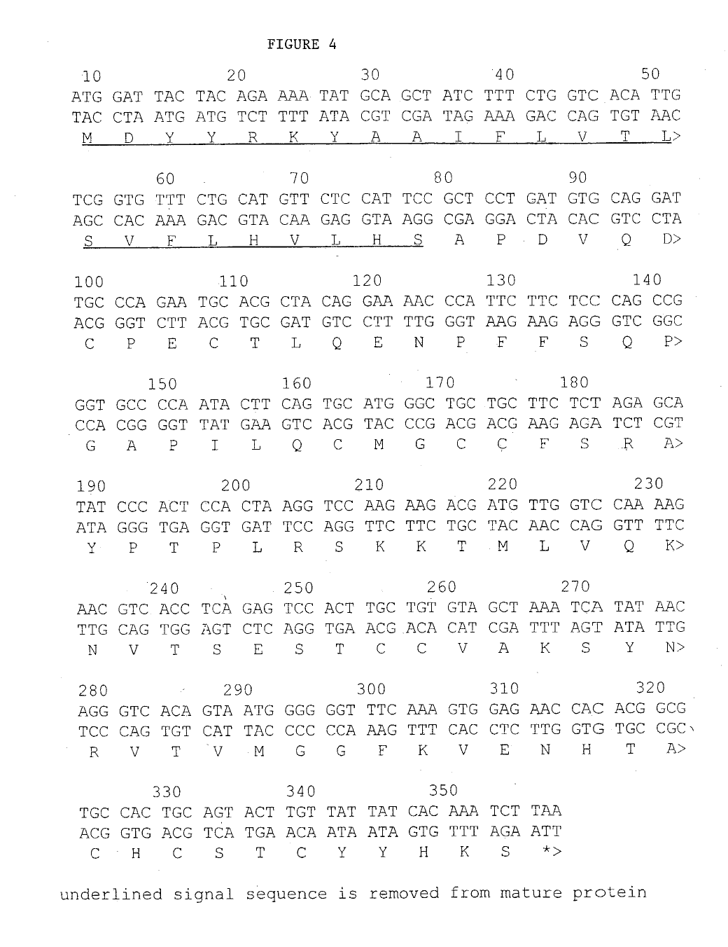

FIG. 5 shows reduced weight of male OGH TG on chow and on high fat diet.

FIG. 6 shows pDEXA analysis which reveals less body fat in male TG after high fat diet.

FIG. 7 shows an increase in relative metabolic rate of OGH TG as compared to wild-type on high fat diet.

FIG. 8 shows improved serum chemistry profile in OGH TG mice.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

Definitions

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the term “OGH-beta-14 mediated condition” is meant a condition which involves the OGH-beta-14 protein. For example, weight loss, obesity and/or metabolic or feeding regulation associated with an increase of OGH-beta-14 are “OGH-beta-14-mediated condition[s].”

A transgenic “knock-in” animal is an animal generated from a mammalian cell which carries a genetic modification resulting from the insertion of a DNA construct targeted to a predetermined, specific chromosomal location which does not alter the function and/or expression of the gene at the site of the targeted chromosomal location. A “knock-out” animal is an animal generated from a mammalian cell which carries a genetic modification resulting from the insertion of a DNA construct targeted to a predetermined, specific chromosomal location which alters the function and/or expression of a gene that was at the site of the targeted chromosomal location. In both cases, the DNA construct may encode a reporter protein such as lacZ, protein tags, and proteins, including recombinases such as Cre and FLP.

This invention is based in part on elucidation of the coding sequence and function of the human protein designated herein as OGH-beta-14. The experiments described below identify functions of OGH-beta-14 to decrease body weight, to decrease body fat, to decrease serum cholesterol, to decrease triglycerides, to decrease blood glucose and to increase adrenal medullary mass. Accordingly, these discoveries provide new methods for the treatment of a variety of maladies including but not limited to: common obesity or undesirable body weight gain, type 1 or type 2 diabetes, hypercholesterolemia, hyperlipidemia and adrenomedullary deficiency; by treatment with OGH-beta-14 protein or functional protein fragment.

Protein and Nucleic Acid Sequence

Applicants have ascertained the amino acid sequence of a novel protein designated herein as OGH-beta-14 (SEQ ID NO: 2 and 9). In addition, isolated nucleic acid sequences are described herein which encode this novel protein or portions thereof. Accordingly, the present invention includes the nucleic acid sequence designated herein as SEQ ID NO: 1 and 8, as well as nucleotide sequences that hybridizes under stringent conditions to the complement of the nucleotide sequence of SEQ ID NO:1 and 8 and which encodes OGH-beta-14, wherein said stringent conditions are 30% formamide in 5×SSPE (0.18 MNaCl, 0.01 MNaPO₄, pH 7.7, 0.001 M EDTA) buffer at a temperature of 42° C. and remaining bound when subject to washing at 42° C. with 0.2×SSPE; and nucleotide sequences which, as a result of the degeneracy of the genetic code, differs from the nucleic acid of SEQ ID NO:3 or sequences which hybridize thereto and which encode OGH-beta-14.

Specific embodiments of the proteins of the invention comprise multimeric versions of the proteins of the invention, e.g., dimers and larger. The multimeric versions may be homo-multimeric and hetero-multimeric. In such instance, the protein of the invention optionally includes a multimerizing component, e.g., a component capable of interacting with a multimerizing component on a second fusion protein to form, for example, a dimer. In one embodiment, the multimerizing component comprises one or more constant region(s) of an immunoglobulin heavy chain. (Takahashi et al. 1982 Cell 29:671-679). In another embodiment, the multimerizing domains is a coiled-coil domain. Such multimerizing component may comprise any other multimerizing component known in the art.

The nucleic acid constructs of the invention are inserted into an expression vector by methods known to the art, wherein the nucleic acid molecule is operatively linked to an expression control sequence. Also provided is a host-vector system for the production of a protein of the invention, which comprises the expression vector of the invention which has been introduced into a host cell suitable for expression of the polypeptide. The suitable host cell may be a bacterial cell such as E. coli, a yeast cell, such as Pichia pastoris, an insect cell, such as Spodoptera frugiperda, or a mammalian cell, such as a COS, CHO, 293, BHK or NS0 cell.

The invention further encompasses methods for producing the proteins of the invention by growing cells transformed with an expression vector under conditions permitting production of the proteins and recovery of the proteins so produced.

The proteins may be purified by any technique which allows for the subsequent formation of a stable protein. For example, and not by way of limitation, the proteins may be recovered from cells either as soluble proteins or protein fragments or as inclusion bodies, from which they may be extracted quantitatively by 8M guanidinium hydrochloride and dialysis. In order to further purify the proteins or fragments, conventional ion exchange chromatography, hydrophobic interaction chromatography, reverse phase chromatography or gel filtration may be used.

Screening and Detection Methods

The proteins of the invention may also be used in in vitro or in vivo screening methods where it is desirable to detect and/or quantify an OGH-beta-14 binding partner. Screening methods are well known to the art that include cell-free, cell-based, and animal assays. In vitro assays can be either solid state or soluble. Detection of bound or complexed protein may be achieved in a number of ways known to the art, including the use of a label or detectable group capable of identifying an agent which has trapped or otherwise bound OGH-beta-14. Detectable labels are well-developed in the filed of immunoassays and may generally be used in conjunction with assays using the protein of the invention.

The proteins of the invention may also be directly or indirectly coupled to a label or detectable group when desirable for the purpose it is being used. A wide variety of labels may be used, depending on the sensitivity required, ease of conjugation, stability requirements, available instrumentation, and disposal provisions

Potential binding compounds can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment, agents that bind OGH-beta-14 are identified in a cell-based assay system. In accordance with this embodiment, cells expressing a OGH-beta-14 protein or protein fragment are contacted with a candidate (or a control compound), and the ability of the candidate compound to bind OGH-beta-14 is determined. The cell may be of prokaryotic origin (e.g., E. coli) or eukaryotic origin (e.g., yeast or mammalian). In specific embodiments, the cell is a OGH-BETA-14 expressing mammalian cell, such as, for example, a COS-7 cell, a 293 human embryonic kidney cell, a NIH 3T3 cell, or Chinese hamster ovary (CHO) cell. Further, the cells may express a OGH-beta-14 protein or protein fragment endogenously or be genetically engineered to express an OGH-beta-14 protein or protein fragment. To identify binding partners for OGH-beta-14, cells expressing a potential binding partner may be screened with OGH-beta-14 utilizing a bioluminescent signal such as the aequorin luminescence assays (see, for example, Button et al. (1993) Cell. Calcium 14:663-671; Liu et al. (1999) Biochem. Biophys. Res. Comm. 266:174-178; Ungrin et al. (1999) Anal. Biochem. 272:34-42; Fujii et al. (2000) J. Biol. Chem 275:21086-21074; Raddatz et al. (2000) J. Biol. Chem. 275:32452-32459; and Shan et al. (2000) J. Biol. Chem. 275:39482-39486, which references are herein specifically incorporated by reference in their entireties). In these binding assays, the OGH-beta-14 is labeled. Cells expressing the test receptor are then incubated with labeled OGH-beta-14, in binding buffer, in cell culture dishes. To determine non-specific binding, unlabeled OGH-beta-14 may be added to the wells. After the incubation, bound and free peptides are separated and detection activity measured in each well.

The binding of the test receptor to OGH-beta-14 can be determined by methods known to those of skill in the art, for example, by flow cytometry, a scintillation assay, immunoprecipitation or western blot analysis. For example, a receptor that binds OGH-beta-14 may be identified using a biological readout in cells expressing an OGH-beta-14 protein or protein fragment. Agonists or antagonists are identified by incubating cells or cell fragments expressing OGH-beta-14 with test compound and measuring a biological response in these cells and in parallel cells or cell fragments not expressing OGH-beta-14. An increased biological response in the cells or cell fragments expressing OGH-beta-14 compared to the parallel cells or cell fragments indicates the presence of a binding partner in the test sample, whereas a decreased biological response indicates absence thereof.

In more specific embodiments, detection of binding and/or modulation of a test agent to a OGH-beta-14 protein may be accomplished by detecting a biological response, such as, for example, measuring Ca²⁺ ion flux, cAMP, IP₃, PIP₃ and transcription of reporter genes. Suitable reporter genes include endogenous genes as well as exogenous genes that are introduced into a cell by any of the standard methods familiar to the skilled artisan, such as transfection, electroporation, lipofection and viral infection.

In another embodiment, agents that bind OGH-beta-14 are identified in a cell-free assay system. In accordance with this embodiment, a OGH-beta-14 protein or protein fragment is contacted with a test (or control) compound and the ability of the test compound to bind OGH-beta-14 is determined. In vitro binding assays employ a mixture of components including an OGH-beta-14 protein or protein fragment, which may be part of a fusion product with another peptide or polypeptide, e.g., a tag for detection or anchoring, and a sample suspected of containing a natural OGH-beta-14 binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature which facilitates optimal binding. The mixture is incubated under conditions whereby the OGH-beta-14 protein binds the test compound. Incubation periods are chosen for optimal binding but are also minimized to facilitate rapid, high-throughput screening.

After incubation, the binding between the OGH-beta-14 protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radiative emissions, nonradiative energy transfers, or indirectly detected with antibody conjugates.

It may be desirable to immobilize either the protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a candidate binding protein and a PGH-beta-14 are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the OGH-beta14, as well as enzyme-linked assays which rely on detecting an enzymatic activity associated with the target molecule.

In another embodiment, agents that bind are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the OGH-beta-14 mediated activity is determined. More specifically, this method may be used to identify an agent capable of modulating OGH-beta-14-mediated conditions.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In one embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a OGH-beta14 mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Cellular Transfection and Gene Therapy

The present invention encompasses the use of nucleic acids encoding the proteins of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.”

In another aspect, the invention provides a method of modulating metabolism and/or reducing weight in a human comprising transfecting a cell with a nucleic acid encoding protein of the invention, wherein the nucleic acid comprises an inducible promoter operably linked to the nucleic acid encoding the protein. For gene therapy procedures in the treatment or prevention of human disease, see for example, Van Brunt (1998) Biotechnology 6:1149-1154.

Combination Therapies

In numerous embodiments, the proteins of the present invention may be administered in combination with one or more additional compounds or therapies. For example, multiple proteins can be co-administered, or one or more proteins can be administered in conjunction with one or more therapeutic compounds. For example, the other therapeutic agent is one used to prevent or treat obesity and/or related diseases, or an agent used to decrease weight.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a transgenic knock-out animal having a modified endogenous OGH-beta14 gene. A transgenic animal can be produced by introducing nucleic acid into the male pronuclei of a fertilized oocyte, e.g., by microinjection, retroviral infection, and allowing the oocyte to develop in a pseudopregnant female foster animal. Still further, the invention contemplates a transgenic animal having an exogenous OGH-beta14 gene generated by introduction of any OGH-beta14-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the OGH-beta-14 protein to particular cells.

It is useful to provide non-human transgenic animals to assay in vivo OGH-beta14 protein function, including receptor interaction, the effect of specific mutant OGH-beta14 proteins on OGH-beta14 protein function and binding partner interaction, and the effect of chimeric OGH-beta14 proteins. It is also possible to assess the effect of null mutations, that is mutations that substantially or completely eliminate one or more OGH-beta14 protein functions.

Therapeutic Uses of Proteins of the Invention

The proteins of the invention can be used to inhibit weight gain, increase lean muscle mass, prevent and treat obesity, and/or modulate feeding and/or metabolism. The proteins of the invention can be used therapeutically or prophylactically in a subject exhibiting or at risk of obesity and related diseases, and/or metabolic or feeding disorders. For example, they can be used to induced weight loss. Further, the proteins of the invention can be used to decrease feeding in the subject. Still further, the proteins can be used prophylactically, e.g. to prevent weight gain, obesity and/or related disorders.

### EXAMPLES

### Example 1

### Identification of HUMAN OGHbeta14[0075]

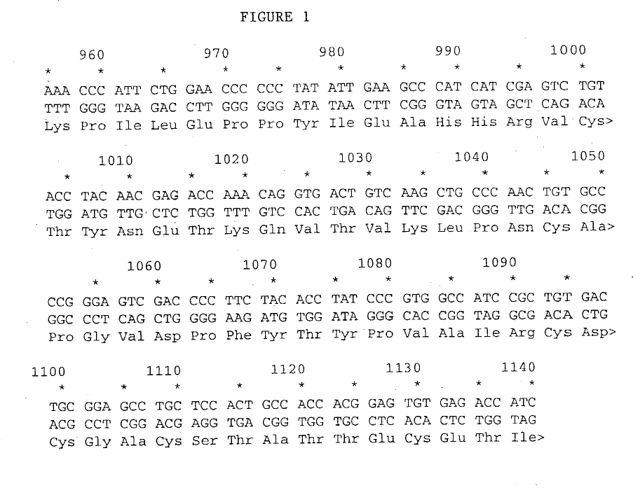

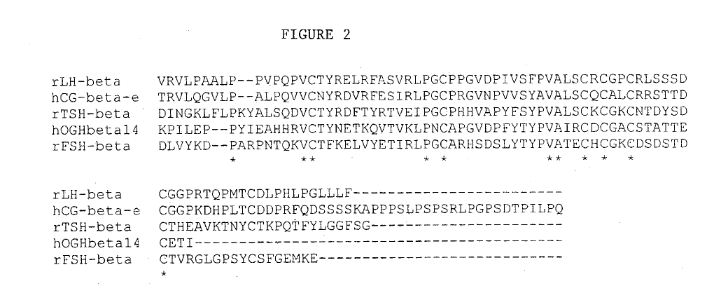

The protein sequence of the beta subunit of human FSH (gi accession number: 476441) was used as an electronic probe to search (using tBLASTn) HTGS, which is that fraction of the GenBank nucleic acid sequence database that contains “unfinished” (phase 1 and phase 2) genomic sequences. Homology was discovered to a region of chromosome 14, clone BAC R-696D21 (accession number: AL049871.1) the nucleic acid sequence of which is set forth in FIG. 1. A conceptual translation of the homologous region revealed a polypeptide sequence termed HUMAN OGHbeta14 (FIG. 1) (SEQ ID NO: 2) that showed homology to each member of the glycoprotein hormone family. The homologous region was also found to have a potential splice acceptor at a location precisely equivalent to introns in each of the other known glycoprotein hormone β subunits, further confirming HUMAN OGHbeta14 as a member of this hormone family. The C-terminus was found to be slightly truncated compared to the other known members, although a potential splice donor site was identified indicating that a longer version might be encoded after splicing to an additional unidentified coding exon. Surprisingly, even though the degree of homology to the other glycoprotein hormone β subunits clearly establishes this DNA sequence as a coding exon, a state-of-the-art computer program designed to discover coding exons in genomic DNA (GenScan) failed to identify HUMAN OGHbeta14 as a coding exon. FIG. 2 (SEQ ID NOs: 3,4,5,6 and 7) is sequence comparison of the novel b subunit polypeptide identified as HUMAN OGHbeta14 with the b subunits of the other glycoprotein hormone family members: rLH-beta, hCG-beta-e, rTSH-beta, and rFSH-beta. Asterisks (*) indicate amino acid identity between all family members.

The protein sequence obtained for HUMAN OGHbeta14 corresponds to amino acid residues 42-103 of hCG-beta (gi accession no. NP_{—}000789). A polypeptide consisting of amino acid residues 38-57 of the hCG-beta (gi accession no. NP_{—}000789) is capable of stimulating steroidogenesis in rat Leydig cells and can strongly inhibit the binding of intact hCG to its receptor (Keutmann, H. T. et al. Proc. Natl. Acad. Sci. USA. 84: 2038-2042).

### Example 2

### Identification of Additional HUMAN OGHbeta14 5′ Sequence

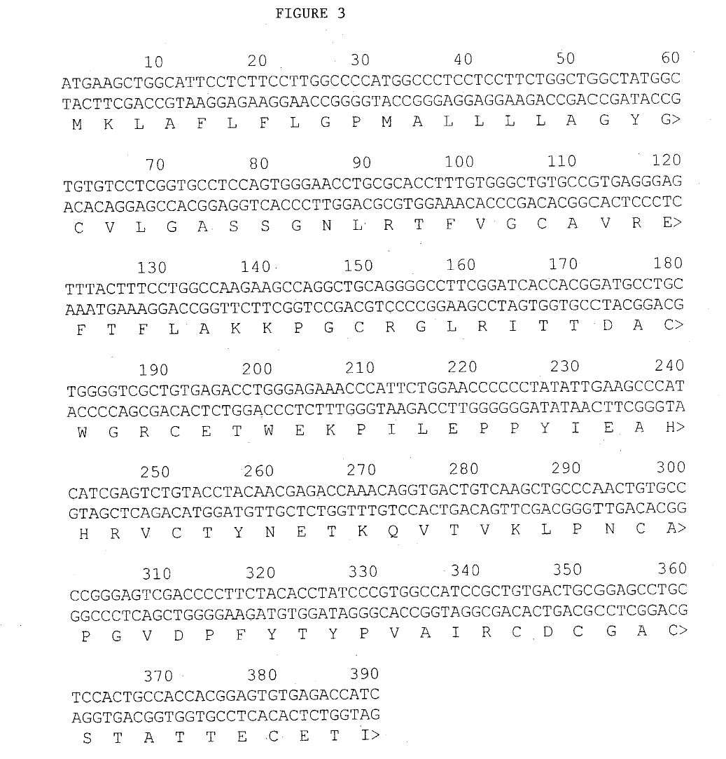

Additional 5′ HUMAN OGHbeta14 sequence was identified as follows. A region of CNS0000U (Accession #ALO498D1.1, GI:4837626, human chromosome 14 bac R-696D21 submitted as phase2 hts sequence on May 14, 1999) corresponding to all unique sequence upstream from the initial predicted exon (FIG. 1) (SEQ ID NO: 2) was analyzed for predicted polypeptides using GENSCAN 1.0. A potential upstream exon containing a predicted signal peptide was identified about 4.5 kb upstream of the initial predicted exon. However this predicted upstream exon contained a predicted splice donor in the wrong reading frame to be spliced directly to the initial predicted exon. The region of the upstream exon was subcloned from a human BAC clone (325d23—Patent Deposit Designation PTA-787) and sequenced. It was determined that the published BAC sequence was in error. The correct sequence contained an additional C (nucleotide 156 in FIG. 3) (SEQ ID NO: 8). Changing the public sequence of . . . CAGGGGCTTCGG . . . (SEQ ID NO 12) to the corrected sequence of . . . CAGGGGCCTTCGG . . . (SEQ ID NO: 13) aligned the putative splice donor and acceptors into the correct reading frame to produce the two exon coding sequence set forth in FIG. 3 (SEQ ID NO: 8). Sequencing of RT-PCR products from pituitary-derived polyA+ RNA confirmed the two exon structure of FIG. 3. After signal peptide cleavage the mature peptide is predicted (SignalP V2.0 World Wide Web Server, http://www.cbs.dtu.dk/services/SignalP-2.0/, Henrik Nielsen, Jacob Engelbrecht, Søren Brunak and Gunnar von Heijne: Identification of prokaryotic and eukaryotic signal peptides and prediction of their cleavage sites. Protein Engineering, 10, 1-6 (1997)) to start at the alanine residue encoded by nucleotides 73 to 75 (GCC) of FIG. 3 (SEQ ID NO: 9).

The nucleotide and deduced amino acid sequence of the extended HUMAN OGHbeta14 sequence is set forth in FIG. 3 (SEQ ID NO: 8 and 9).

This novel protein may function as a heterodimer with the known common glycoprotein hormone alpha (α) subunit, a novel undiscovered alpha (α) subunit or as a monomer or homodimer. The latter two possibilities are suggested by the relatively premature termination of HUMAN OGHbeta14 compared to the other four glycoprotein hormone β subunits. There is no cysteine residue at a position homologous to either Cys26 or Cys110 of the hCG sequence. These residues have been found to be necessary to form the “seat belt” in the crystal structure of hCG (Lapthom, A. J., et al. Nature 369:455-461; Wu, H., et al. Structure 1:153-159). This structure is responsible for stabilizing the heterodimer. Indeed we have found that HUMAN OGHbeta14 can form both homodimers as well as heterodimers with the common glycoprotein hormone alpha (a) subunit. Both of these structures are stable in that there is no association of differentially epitope tagged HUMAN OGHbeta14 or α subunits if they are mixed post-synthetically.

### Example 3

### Expression of HUMAN OGHbeta14

TaqMan analysis: The abundance of mRNA was determined using the quantitative RT-PCR “TaqMan” procedure (Lie, Y. S. and C. J., Petropoulos, “Advances in quantitative PCR technology: 5′ nuclease assays”, Curr Opin Biotechnol 9 (1998): 43-48.) with a PE ABI PRISM 7700 Sequence Detection System instrument (PE Biosystems, Foster City, Calif.).

This method employs two oligonucleotides spaced relative close to each other to PCR amplify a portion of the message from cDNA and a third “probe” oligonucleotide co-labeled with a fluorophore and quencher at each end. When the level of the PCR product builds up to a sufficient level, a significant fraction of the fluorophore is released by a “nick-translation” exonucleolytic activity of the polymerase. The released fluorophore becomes highly fluorescent by being dissociated from the quenching moiety. The abundance of a specific mRNA is determined by reading fluorescence during the course of the PCR reaction: samples containing more abundant messages taking fewer PCR cycles to release probe fluorescence, while samples containing the same message in lower abundance will require more cycles.

For TaqMan analysis of HUMAN OGHbeta14 the following oligonucleotides were employed:

hOGH(B)for: TACTTTCCTGGCCAAGAAGCC (SEQ ID NO: 14). hOGH(B)rev: CACCTGTTTGGTCTCGTTGTAG (SEQ ID NO: 15). hOGH(B)TaqMan: AGACCTGGGAGAAACCCATTCTGGAAC (SEQ ID NO: 16). The TaqMan PCR reactions were run on a Perkin Elmer ABI PRISM 7700 Sequence Detection System instrument. MicroAmp (Perkin Elmer) optical 96-well plates and optical caps were used. Each reaction had a final volume of 25 μl and the following concentrations of components: 1× TaqMan buffer A, 4 mM MgCl_{2, 200} mM of each of dATP, dCTP, dGTP, and 400 mM dUTP, 300 nM of each of forward (hOGH(B)for) and reverse (hOGH(B)rev) primers, 200 nM of hOGH(B)TaqMan probe, 5% DMSO, 10% glycerol, 0.025 U/ml AmpliTaq Gold, 1U/ml AmpErase UNG and oligo-dT-primed cDNA derived from either 50 ng of total RNA or the indicated amount of poly a+ RNA. The PCR cycling conditions were as follows: 2 min. at 50° C., 10 min. at 95° C., followed by 40 two-step cycles of 15 sec at 95° C. and 1 min. at 60° C. The TaqMan probe had a 6-FAM 5′-Fluorescent label and TAMRA 3′-label that acts as a quencher.

The analysis, shows weak expression of HUMAN OGHbeta14 in pituitary, testis, thymus and trachea. The expression levels are normalized to molecules of mRNA per cell assuming each cell contains 20 pg of total RNA or 1 pg of poly A+ RNA

Northern analysis: Northern blot analysis was carried out using 5 μg of human poly A+ RNA per lane. RNA was purchased from Clonetech. The probe was a fragment containing the entire HUMAN OGHbeta14 coding sequence, and exposure times were 7 days.

There was expression of HUMAN OGHbeta14 using Northern analysis. The results of this analysis reveal the presence of a 1.3 kb transcript in the pituitary. Since the pituitary is the site of expression of the homologous glycoprotein hormone family members LH, FSH and TSH, it seems likely that HUMAN OGHbeta14 is also a circulating hormone.

### Example 4

### Subunit Structure of HUMAN OGHbeta14

Epitope tagged versions of the indicated proteins HUMAN OGHbeta14, hCG (human Chorionic Gonadotropin beta subunit; Genebank acc#NP_{—}000728) and alpha (a) (the human, common alpha subunit of the glycoprotein hormones; Genebank acc#NP_{—}000726) were expressed in COS cells using standard procedures known to the skilled artisan.

Media supernatants obtained from the COS transfectants described supra were either run directly (15 μl per lane, Panel A and Panel B) on reducing, denaturing acrylamide gels (4-20% gradient, Novex) or run after immunoprecipitation (Panel C) as follows: One ml of culture supernatant from each transfection was chilled on ice and mixed with 0.5 ml of cold TBS, 2.2 μg of M2 anti-FLAG monoclonal antibody (Sigma) and 0.05 ml of protein G sepharose beads (Pharmacia). The mixture was gently mixed at 4° C. for 2.5 hours. The beads were collected by centrifugation, washed 3 times with TBS plus 1% NP40 and proteins were eluted from pelleted beads with 30 μl of loading buffer. 15 μl of the recovered proteins were loaded per gel lane.

Gels were transferred overnight by standard procedures, blocked with 10% non-fat dried milk and probed with either anti-HA (0.5 μg/ml monoclonal 12CA5, Boehringer-Mannheim) or anti-FLAG (0.44 μg/ml monoclonal M2, Sigma) for 1 hour, washed, probed with a secondary antibody (0.077 μg/ml HRP conjugated anti-mouse IgG, Promega), washed and developed with the ECL luminescence kit as per manufacturers instructions (NEN).

Flag-tagged HUMAN OGHbeta14 immunoprecipitated both co-expressed HA-tagged common glycoprotein hormone alpha (a) subunit and HA-tagged HUMAN OGHbeta14 with efficiencies roughly comparable to that at which FLAG-tagged hCG immunoprecipitated common glycoprotein hormone alpha (a) subunit. These results indicate that HUMAN OGHbeta14 can form homo-dimers (or higher order homomeric structures) as well as hetero-dimers (or higher order heteromeric structures) with the common glycoprotein hormone alpha (a) subunit. The homo-multimeric and hetero-multimeric forms are likely to have different biological activities perhaps binding to different receptors or acting as an agonist/antagonist pair on the same receptor or group of receptors.

### Example 5

### OGH TG Mice

Mice were constructed (hereafter referred to as OGH TG mice) in which the OGHbeta14 gene is inserted into the ubiquitously expressed Rosa26 locus, in such a way that its expression is driven from the ubiquitously expressed Rosa26 promoter (REG781). These mice displayed several beneficial phenotypes compared to their wild type littermates.

Necropsy and histopathology were performed on several animals and it was determined that there was a tendency for OGH TG mice to have lower amounts of visible body fat, whereas all of the OGH TG mice, and none of their wild type litter mates, showed enlarged adrenal medullas, characterized by moderate hyperplasia, hypertrophy and intracellular lipidosis.

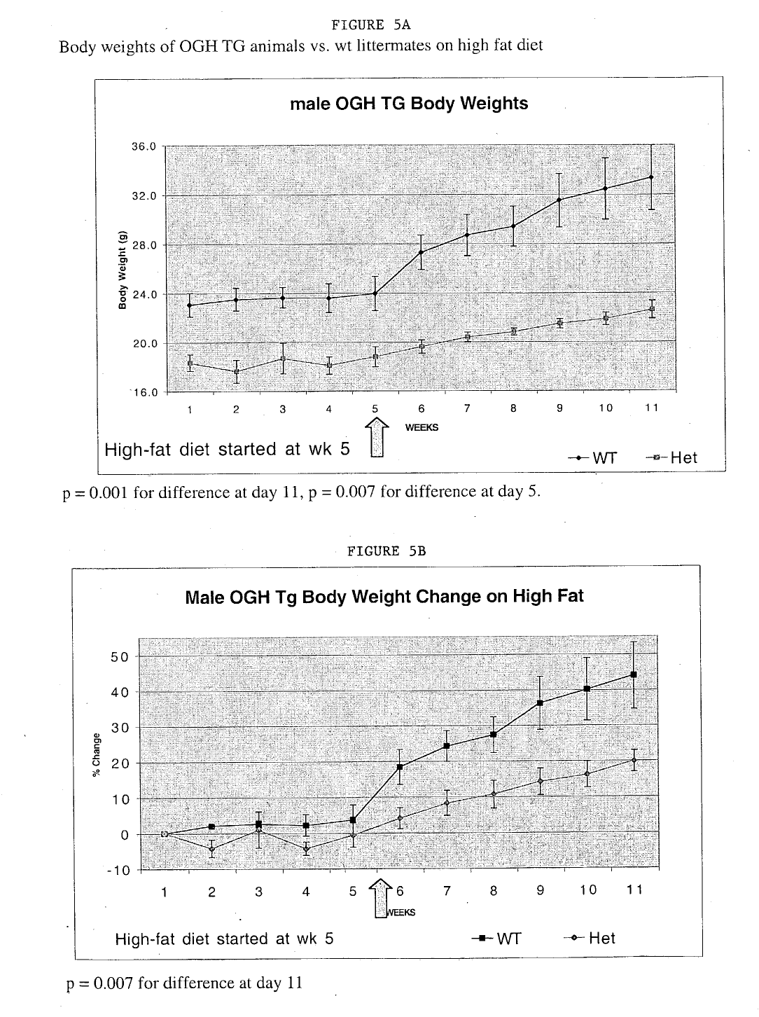

Male OGH TG animals showed significantly lower body weights when fed either normal or high-fat chow, and significantly less proportional weight gain on a high fat diet (FIG. 5). Weight gain on a high-fat diet is a model for common human obesity. Wild type females of this mouse background gain much less weight when placed on a high-fat diet than do the males, so most of our analyses were done with male animals.

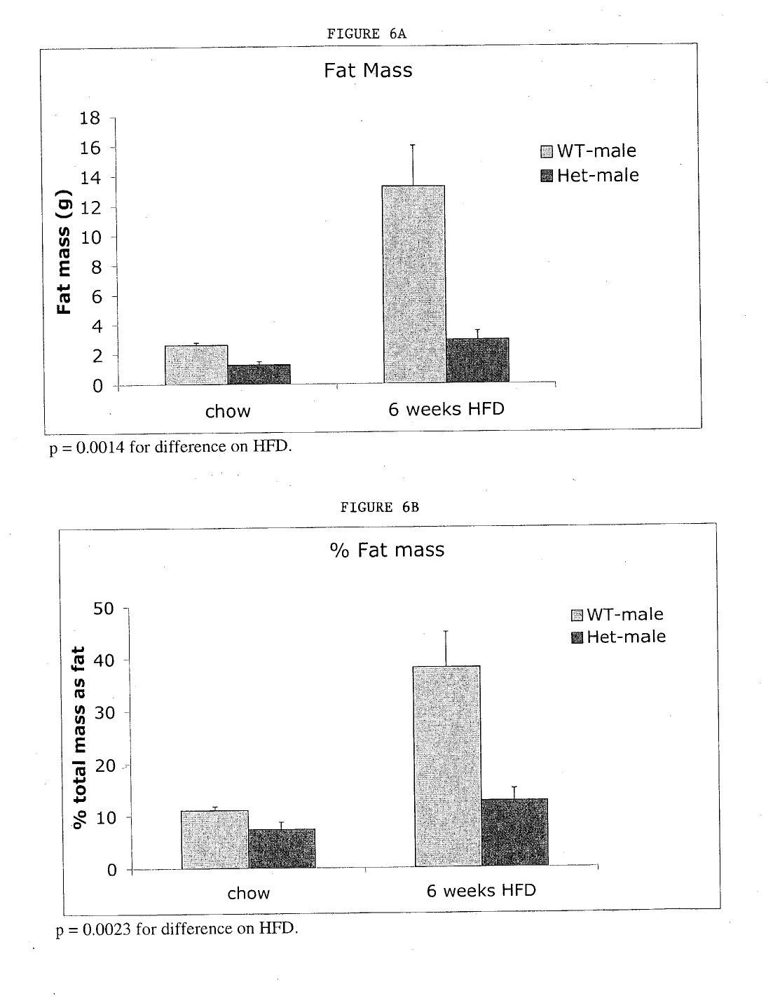

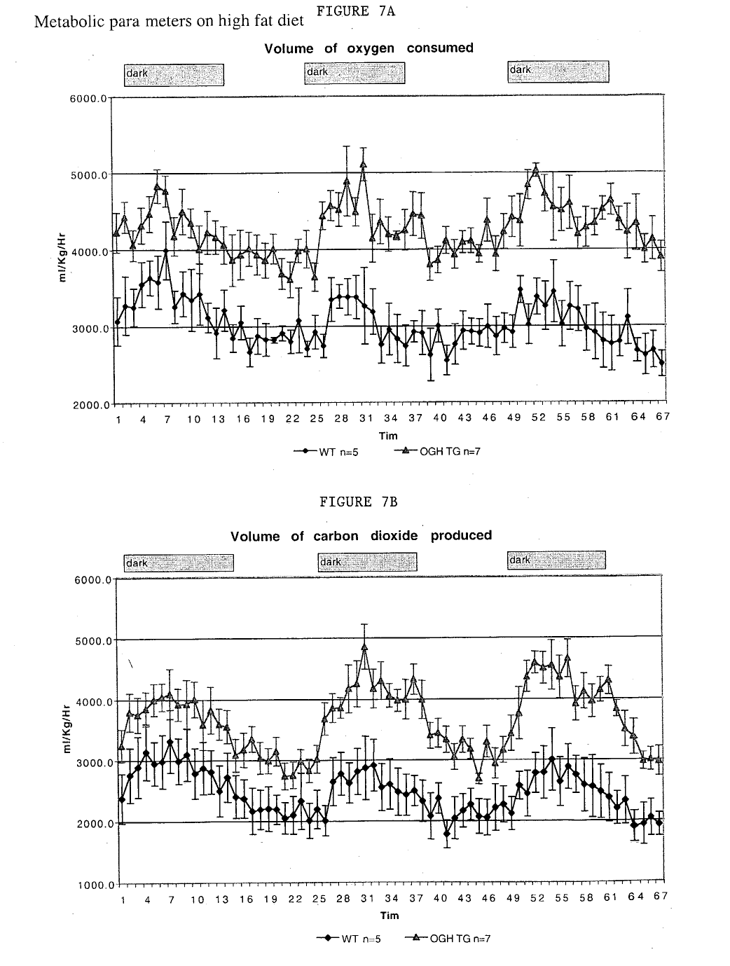

Body composition measurements were made on age matched OGH TG and wild type mice before and after a high-fat diet using dual-energy X-ray absorptiometry (pDEXA, Nagy TR and Clair AL; Obes Res. 2000 5:392-8; Sjogren K. et al.; J Nutr. 2001 11:2963-6.). OGH TG animals showed significantly less body fat after a high-fat diet than wild type animals (FIG. 6). At least part of the lower body weight and lower body fat in the OGH TG mice is due to a higher metabolic rate, as measured by oxygen consumed and carbon dioxide produced, of these animals on a high-fat diet (FIG. 7).

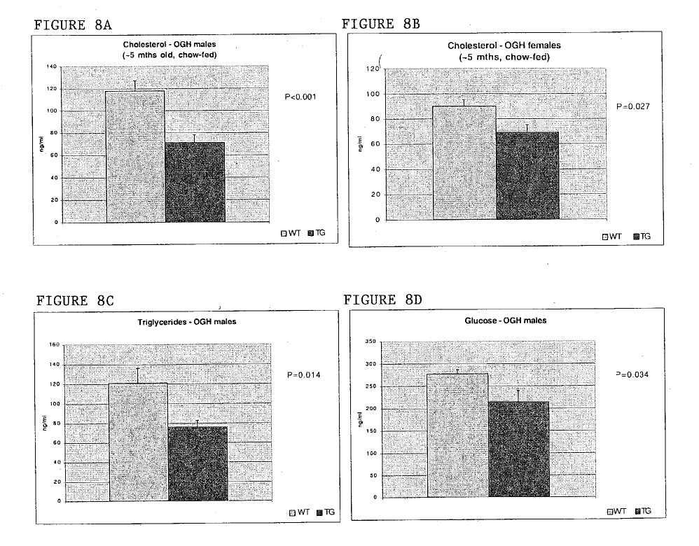

In addition to their resistance to weight gain on a high-fat diet, the OGH TG mice showed a “healthier” serum chemistry profile in older mice on normal chow (FIG. 8). Both males and females had significantly lower serum cholesterol, and males had significantly lower blood levels of triglycerides and glucose. Triglyceride and glucose levels are elevated in type 1 and type 2 diabetes, and an agent that lowered them would be useful in treating these diseases.

Deposit of Biological Material

The following clones were deposited with the American Type Culture Collection ATCC®), 10801 University Boulevard, Manassas, Va. 20110-2209, on Sep. 24, 1999:

| Clone | Patent Deposit Designation |
|---|---|
| 325d23 human DNA insert in BAC vector | PTA-787 |
| 534i21 human DNA insert in BAC vector | PTA-788 |
| 399n04 human DNA insert in BAC vector | PTA-789 |

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A therapeutic method for decreasing body weight, treating obesity, lowering serum cholesterol, lowering serum triglycerides, lowering blood glucose and/or modulating metabolism in a subject comprising administering a therapeutically effective amount of OGHbeta14 protein, or protein fragment to an animal.

2. The method of claim one wherein said animal is a human.

3. A pharmaceutical composition useful for decreasing weight, treating obesity, and/or modulating feeding or metabolism comprising OGHbeta14 and a pharmaceutical acceptable carrier.

4. A transgenic animal, comprising a modification of an endogenous and/or exogenousOGHbeta14 gene.

5. A transgenic animal, comprising a human OGHbeta14 gene.

6. An isolated OGHbeta14 polypeptide.

7. An isolated OGHbeta14 polypeptide comprising the amino acid sequence as set forth in SEQ ID NO: 2 or 9.

8. An isolated OGHbeta14 polypeptide encoded by the nucleic acid molecule as set forth in SEQ ID NO: 1 or 8.

9. An isolated nucleic acid molecule having a sequence selected from the group consisting of:
(a) the nucleotide sequence comprising the coding region of the OGHbeta14 as set forth in SEQ ID NO: 1; or
(b) a nucleotide sequence that hybridizes under stringent conditions to the complement of the nucleotide sequence of (a) and which encodes OGHbeta14, wherein said stringent conditions are 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE; or
(c) a nucleotide sequence which, as a result of the degeneracy of the genetic code, differs from the nucleic acid of (a) or (b) and which encodes OGH-beta14.

10. A method of producing OGHbeta14 which comprises growing cells of an OGH-beta14 host-vector system, under conditions permitting production of the OGH-beta14, and recovering the OGH-beta14 so produced.

11. A method for identifying a receptor capable of binding a OGHbeta14 protein, or protein fragment, comprising:
(a) contacting a test receptor with a OGHbeta14 protein, or protein fragment; and
(b) determining the ability of the test receptor to bind OGHbeta14 protein or protein fragment.

12. The method of claim 14, wherein the receptor is a GPCR.

13. The method of claim 14, wherein the OGHbeta14 protein comprises the amino acid sequence of SEQ ID NO:2 or 9, or a fragment thereof.

14. The method of claim 14, wherein the receptor is involved in metabolism or feeding.
